(19) United States
(12) Patent Application Publication
(10) Pub. No.: US 2017/0326105 A1
(43) Pub. Date: Nov. 16, 2017

(54) MELATONIN-BASED NASAL COMPOSITIONS AND USES THEREOF

(71) Applicant: The Lester Smith Medical Research Institute, LLC, Houston, TX (US)

(72) Inventor: Ramon Coronado, San Antonio, TX (US)

(73) Assignee: The Lester Smith Medical Research Institute, LLC, Houston, TX (US)

(21) Appl. No.: 15/592,005

(22) Filed: May 10, 2017

Related U.S. Application Data
(60) Provisional application No. 62/334,122, filed on May 10, 2016.

(51) Int. Cl. A61K 31/405; A61K 31/4045; A61K 9/00; A61K 47/40

(52) U.S. Cl. CPC A61K 31/405; A61K 47/40; A61K 9/0043; A61K 9/0073; A61K 31/4045

(57) ABSTRACT

Disclosed is an improved composition characterized in containing a combination of melatonin and 5-HTP in a nasal administration for the rapid treatment of insomnia, anxiety, and related disorders in mammals, including humans. The present invention relates also to a pharmaceutical composition comprising 5-HTP and melatonin which utilizes the known effects of two substances to provide sleep improvement, relaxation, and reduced anxiety via rapid delivery of the combination, via nasal administration, for short-term alleviation of sleep disorder and anxiety-related symptoms. The combined effects of 5-HTP and melatonin in the present invention provide a short-term increase in blood plasma levels of melatonin while simultaneously increasing the production of serotonin for purposes of achieving natural melatonin biosynthesis in combination with the increased melatonin content in blood plasma.

### CROSS-REFERENCE TO RELATED APPLICATIONS

This application claims priority to U.S. Patent Application Ser. No. 62/334,122, filed May 10, 2016, entitled “Melatonin-Based Nasal Composition and Uses Thereof”. The foregoing patent application is hereby incorporated by reference in its entirety for all purposes.

This application includes material that is subject to copyright protection. The copyright owner has no objection to the facsimile reproduction by anyone of the patent disclosure, as it appears in the Patent and Trademark Office files or records, but otherwise reserves all copyright rights whatsoever.

### TECHNICAL FIELD

The present invention relates in general to the field of prevention and treatment of sleep-related and anxiety disorders. In particular, the present invention provides for a melatonin-based nasal delivery composition and methods of use thereof. The disclosed systems and methods support a variety of scenarios for treatment of sleep disorders, anxiety, and for related products and services.

### STATEMENT OF FEDERALLY FUNDED RESEARCH

None.

### BACKGROUND OF THE DISCLOSURE

Melatonin, chemically N-acetyl-5-methoxy tryptamine, is a substance found in animals, plants, fungi, and bacteria. In animals, it is a hormone that anticipates the daily onset of darkness. In animals, melatonin is involved in the entrainment (synchronization) of the circadian rhythms of physiological functions including sleep timing, blood pressure regulation, seasonal reproduction and many others. Many of melatonin's biological effects in animals are produced through activation of melatonin receptors, while others are due to its role as a pervasive and powerful antioxidant, with a particular role in the protection of nuclear and mitochondrial DNA.

Melatonin can be used as a sleep aid and in the treatment of some sleep disorders. It can be taken orally in liquid form as well as capsules or tablets in immediate- or prolonged-release form. It is also available in a form to be used sublingually, and as transdermal patches.

In exploratory studies, prolonged-release melatonin has shown sleep quality improvement in patients with schizophrenia as well as in patients with major depressive disorder. It has shown promise in treating sleep-wake cycle disorders in children with underlying neurodevelopment difficulties. Additionally, as add-on to antihypertensive therapy, prolonged-release melatonin improved blood pressure control in patients with nocturnal hypertension as shown in studies.

5-HTP (L-5 Hydroxytryptophan) is a natural precursor to serotonin and thereby supports healthy levels of this important neurotransmitter. The pineal gland is governed by the circadian rhythm, producing serotonin during daylight hours then shifting to melatonin throughout the evening and night. Some therapies utilize both melatonin and 5-HTP, as combining 5-HTP with pure melatonin provides a two-tier approach to healthy sleep, supporting natural neurotransmitter production.

While combinations of melatonin and 5-HTP are known, risks of such combination products involve delayed effects and side effects following sleep, such as grogginess, lethargy or hangover effects. Many of these treatments do not take advantage of the multiple metabolic pathways used by these compounds. Despite advances in the art, there remains a need to improve utilization of such products by allowing for beneficial results while reducing possible negative effects.

### SUMMARY OF THE DISCLOSURE

The present invention addresses limitations in the art by providing an improved composition that is characterized in containing a combination of melatonin and 5-HTP. Particularly, the present invention relates to the combined use of 5-HTP and melatonin in a nasal administration for the rapid treatment of insomnia, anxiety, and related disorders in mammals, including humans. The invention relates also to a pharmaceutical composition comprising 5-HTP and melatonin. A combined composition utilizes the known effects of two substances to provide sleep improvement, relaxation, and reduced anxiety. Further aspects include rapid delivery of the combination, via nasal administration, for short-term alleviation of sleep disorder and anxiety-related symptoms.

It is therefore an object of the present invention to provide a pharmaceutical composition for intranasal administration, comprising: 5-HTP; melatonin; and a pharmaceutically acceptable carrier. It is another object of the present invention to provide a method of rapid improvement of sleep behavior of a subject, comprising administering to a patient via nasal administration a therapeutically effective amount of a composition comprising 5-HTP and melatonin.

In one aspect, the present invention provides a fast-acting composition for intranasal administration to a patient, comprising: 5-hydroxytryptophan (5-HTP); melatonin; and a pharmaceutically acceptable carrier, wherein said composition is capable composition being effective to cause the blood plasma concentration of melatonin in a patient to rapidly increase while simultaneously causing an increase in 5-HTP present in central nervous system tissue to increase natural production of melatonin.

In another aspect, the composition is capable of affecting a patient in 30, 15, 10 minutes or less. The 5-HTP may be administrable in a range of 0.1 μg-650 mg, and the melatonin is administrable in a range of 0.1 μg-500 mg. The pharmaceutically acceptable carrier is selected from the group consisting of water, saline, cyclodextrin, glycerol, or combinations thereof. In one aspect, the pharmaceutically acceptable carrier is β-cyclodextrin. In another aspect, the pharmaceutically acceptable carrier is: α-cyclodextrin, β-cyclodextrin, γ-cyclodextrin, methylated β-cyclodextrin, hydroxypropyl- and hydroxyethyl-cyclodextrin (di)glucosyl- or (di)maltosyl-cyclodextrins, carboxymethyl-cyclodextrins, or sulfobutylether-β-cyclodextrin.

The intranasal administration may be a spray, drop, solution, suspension, gel, ointment, cream, powder, nasal tampon or a nasal sponge.

In one aspect of the present invention, the composition is utilized for intranasal administration for rapid treatment of insomnia. In another aspect the composition is utilized for intranasal administration for rapid treatment of anxiety.

It is another object of the present invention to provide a method of rapid improvement of sleep behavior of a patient, comprising administering to a patient via nasal administration a therapeutically effective amount of a composition comprising 5-HTP and melatonin, wherein said composition causes the blood plasma concentration of melatonin in a patient to rapidly increase while simultaneously causing an increase in 5-HTP present in central nervous system tissue to increase natural production of melatonin. In one aspect the composition further comprises a pharmaceutically acceptable carrier, which may be comprised of water, saline, cyclodextrin, glycerol, or combinations thereof. The composition may be administered in the form of a spray, drop, solution, suspension, gel, ointment, cream, or powder, nasal tampon or a nasal sponge.

It is another object of the present invention to provide a method of treating anxiety of a subject, comprising administering to a patient via nasal administration a therapeutically effective amount of a composition comprising 5-HTP and melatonin, wherein said composition causes the blood plasma concentration of melatonin in a patient to rapidly increase while simultaneously causing an increase in 5-HTP present in central nervous system tissue to increase natural production of melatonin. The composition may further comprise a pharmaceutically acceptable such as water, saline, cyclodextrin, glycerol, or combinations thereof. Additionally, the composition may be administered in the form selected from the group consisting of a spray, drop, solution, suspension, gel, ointment, cream, or powder, nasal tampon or a nasal sponge.

### BRIEF DESCRIPTION OF THE DRAWINGS

The foregoing and other objects, features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 depicts the chemical formula of melatonin.

FIG. 2 depicts the chemical formula for 5-HTP

FIG. 3 depicts the biological metabolism of tryptophan to melatonin.

FIG. 4 depicts a chart showing respondent patient (n=12) survey data regarding how many nostrils was the composition spray administered to.

FIG. 5 depicts a chart showing respondent patient (n=11) survey data regarding how many sprays were applied per nostril.

FIG. 6 depicts a chart showing respondent patient (n=11) survey data regarding whether an effect was felt by the patient.

FIG. 7 depicts a chart showing respondent patient (n=11) survey data regarding the nature of the effect.

FIG. 8 depicts a chart showing respondent patient (n=11) survey data regarding the amount of time until the effect was felt.

FIG. 9 depicts a chart showing respondent patient (n=11) survey data regarding the amount of time the effect lasts.

FIG. 10 depicts a chart showing respondent patient (n=11) survey data regarding the nature of the effect applied to improved sleep.

FIG. 11 depicts a chart showing respondent patient (n=11) survey data regarding the nature of the effect applied to improved relaxation.

FIG. 12 depicts a chart showing respondent patient (n=11) survey data regarding the lessening of any symptoms.

FIG. 13 depicts a chart showing respondent patient (n=11) survey data regarding the rapid onset of the present invention when compared to oral medications/supplements.

### DETAILED DESCRIPTION OF THE DISCLOSURE

While the making and using of various embodiments of the present invention are discussed in detail below, it should be appreciated that the present invention provides many applicable inventive concepts that can be embodied in a wide variety of specific contexts, goods, or services. The specific embodiments discussed herein are merely illustrative of specific ways to make and use the disclosure and do not delimit the scope of the disclosure.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The present invention will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The biosynthesis of melatonin is initiated by the uptake of the essential amino acid tryptophan into pineal parenchymal cells. Tryptophan is the least abundant of essential amino acids in normal diets. It is converted to another amino acid, 5-hydroxytryptophan (5-HTP), through the action of the enzyme tryptophan hydroxylase and then to 5-hydroxytryptamine (serotonin) by the enzyme aromatic amino acid decarboxylase.

Serotonin is a neurotransmitter and is found in all bilateral animals, where it mediates gut movements and the animal's perceptions of resource availability. In less complex animals, such as some invertebrates, resources simply mean food availability. In more complex animals, such as arthropods and vertebrates, resources also can mean social dominance. In response to the perceived abundance or scarcity of resources, an animal's growth, reproduction or mood may be elevated or lowered. This may somewhat depend on how much serotonin the organism has at its disposal.

Serotonin concentrations are higher in the pineal than in any other organ or in any brain region. They exhibit a striking diurnal rhythm remaining at a maximum level during the daylight hours and falling by more than 80% soon after the onset of darkness as the serotonin is converted to melatonin, 5-hydroxytryptophol and other methoxyindoles. Serotonin's conversion to melatonin involves two enzymes that are characteristic of the pineal: SNAT (serotonin-N-acetyltransferase) which converts the serotonin to N-acetylserotonin, and HIOMT (hydroxyindole-O-methyltrasferase) which transfers a methyl group from S-adenosylmethionine to the 5-hydroxyl of the N-acetylserotonin. The activities of both enzymes rise soon after the onset of darkness because of the enhanced release of norepinephrine from sympathetic neurons terminating on the pineal parenchymal cells.

Another portion of the serotonin liberated from pineal cells after the onset of darkness is deaminated by the enzyme monoamine oxidase (MAO) and then either oxidized to form 5-hydroxyindole acetic acid or reduced to form 5-hydroxytryptophol. Both of these compounds are also substrates for HIOMT and can thus be converted in the pineal to 5-methoxyindole acetic acid 5-methoxytryptophol. The level of this latter indole, like that of melatonin, rises markedly in the pineal with the onset of darkness. Since 5-methoxytryptophol synthesis does not require the acetylation of serotonin, the nocturnal increase in pineal SNAT activity cannot be the trigger that causes pineal methoxyindole levels to rise. More likely, a single unexplained process—the intraparenchymal release of stored pineal serotonin, which then becomes accessible to both SNAT and MAO. This process ultimately controls the rates at which all three major pineal methoxyindoles are synthesized and generates the nocturnal increases in pineal melatonin and 5-methoxytryptophol. The proportion of available serotonin acetylated at any particular time of day or night depends on the relative activities of pineal SNAT and MAO at that time. The rates of methylation of all three 5-hydroxyindoles formed from pineal serotonin depend on HIOMT activity.

5-Hydroxytryptophan (5-HTP), also known as oxitriptan (INN), is a naturally occurring amino acid and chemical precursor as well as a metabolic intermediate in the biosynthesis of the neurotransmitters serotonin and melatonin from tryptophan. The psychoactive action of 5-HTP is derived from its increase in production of serotonin in central nervous system tissue.

Melatonin is usually administered orally but, as with most oral preparations, it takes over an hour after administration for the blood plasma concentration of the active agent (melatonin) to reach its peak. Other routes of administration, in particular nasal administration have been considered. However, because of inherent problems, inter alia due to the low solubility of melatonin in water, melatonin compositions suitable for nasal administration have yet to be considered commercially acceptable for patient use or consumption.

For those melatonin compositions that are available for use, most focus on the increase of the blood plasma concentration of melatonin to achieve desired results. However, these compositions are not indicative of the biosynthetic pathway of melatonin, which relies on the synthesis of melatonin via the metabolic effects of the increased levels of serotonin. Serotonin, in turn, is then metabolized into melatonin, in connection with N-acetyltransferase (NAT), converting serotonin to N-acetylserotonin (NAS) and subsequently to melatonin by hydroxyindol-O-methyl transferase (HIOMT),

It is therefore an exemplary embodiment of the present invention to provide a composition for intranasal administration comprising a combination of 5-HTP and melatonin and a pharmaceutically acceptable excipient, said composition being effective to cause the blood plasma concentration of melatonin in a human to increase while simultaneously causing an increase in 5-HTP present in central nervous system tissue to increase natural production of serotonin. The combined effects of the dually administered ingredients provide a rapid increase in melatonin within the blood plasma via nasal administration while triggering increased serotonin production by administering the 5-HTP within the nasal administration. The dual effects of the two ingredients result in a rapid yet short term increase in melatonin and serotonin production, which enhances the anti-anxiety and anti-insomnia effects of the administered compositions.

In one embodiment, the present invention provides, in a first aspect, a pharmaceutical composition for intranasal administration, comprising melatonin and a pharmaceutically acceptable carrier, characterized by being effective to cause the blood plasma melatonin concentration in a human adult, receiving an amount of melatonin in the range of 0.1 μg-650 mg and an amount of 5-HTP in the range of 0.1 μg-500 mg in a single or simultaneous intranasal administration of said composition. In general, the total daily dose range is from about 1 mg to about 300 mg. Dosage amounts vary according to a patient's age, body weight, and response of the individual patient. Doses ranges may be ideally administered daily between about 1 mg to about 150 mg. Preferably, doses may range from 3 mg to 70 mg. In certain embodiments, a daily dosage of 1, 3, 5, 10, 20, 30, or 40 mg may be preferred depending upon patient response. In managing the patient, the therapy may be initiated at a lower dose, perhaps about 3 mg to about 8 mg and increased up to about 10 mg or higher depending on the patient's overall response.

In one embodiment of the present invention the following materials may be incorporated into a composition for nasal administration: melatonin, 5-HTP, β cylcodextrin, glycerol, sorbitol, and benzalkonium chloride, incorporated into the nasal solution in various amounts. In an exemplary embodiment, a composition of the present invention comprises a nasal spray formulation having: melatonin-1 mg/mL; 5-HTP-13.5 mg/mL; βcyclodextrin-5 mg/mL; glycerol: 0.1 mg/mL; sorbitol: 50/mL; and benzalkonium chloride: 0.1 mg/mL. In another embodiment of the present invention, additional compositions include GABA: 50 mg/mL; Vitamin B12: 4 mg/mL; and DMSO.

In another embodiment of the present invention, the melatonin and 5-HTP concentrations are reached within 15, 10, 5 or 2 minutes of administration. Compositions in accordance with this aspect of the invention can also comprise a saccharide, a polysaccharide or a triol. Melatonin-based compositions may comprise one or more melatonin agents, including melatonin, selective melatonin agonists, melatoninergic agonists such as agomelatine, melatonin receptor agonists, melatonin analogs, controlled release melatonin, competitive antagonists of melatonin receptors, melatonin analogues, and other osmotic systems for melatonin deliver. In another embodiment, the melatonin is a pharmaceutically acceptable salt, solvate, clathrate, polymorph, or co-crystal thereof.

In another embodiment, the 5-HTP is obtained via an extract from Griffonia simplicifolia, Mucana pruriens of legume, Musa sapientum, Gossypium hirsutum, and/or Urtica dioica. The 5-HTP can be more than about 0.01 percent of the composition. The 5-HTP can contain optically pure L-configuration 5-HTP, and may further be comprised of a pharmaceutically acceptable salt, solvate, clathrate, polymorph, or co-crystal thereof.

In further embodiments, compositions in accordance with the present invention, when intranasally administered, are effective to cause a rapid peak blood plasma melatonin concentration, or tₘₐₓ within 30 minutes of administration and, preferably, within 15 or 10 minutes of administration. In a preferred embodiment, the rapid peak blood plasma melatonin and 5-HTP concentrations are reached within 5 minutes of administration.

The “blood plasma melatonin concentration” can be a mean value measured in a study of the type described herein, and in such studies or trials that are well known to those skilled in the art. Similarly, the “blood plasma 5-HTP concentration” may be similarly measured.

The plasma half-life is short, 20 to 50 minutes, and plasma levels return to baseline within 24 hours after discontinuation of chronic dosing of less than 10 mg/day. Doses of melatonin 5 mg produce estimated peak blood levels 25 times above physiological levels, but do not alter endogenous melatonin production.

When used in this specification, the term “single or simultaneous intranasal administration” encompasses both the administration of a single dose or dose form, such as a single insulation of a powder, or a single application of a spray, and the contemporaneous administration of a plurality of such doses or dose forms, (for example, the administration of two squirts of a spray or powder insufflations, one in each nostril).

It has also been found that glycerol and cyclodextrin, when employed in nasal compositions containing melatonin, do not exhibit the unwanted toxic and adverse effects previously noted with the use of (poly)alcohols. It has further been found that cyclodextrin accelerates the absorption of melatonin in the nasal mucosa. In another embodiment, a solution of melatonin, 5-HTP, and a liquid carrier such as water or saline is used. In a further embodiment, preservatives may be used or the solution is preservative free, and may be further acidified.

Thus, in a second aspect, the present invention provides a pharmaceutical composition for intranasal administration, comprising a combination of melatonin, 5-HTP, and an additive, wherein the additive comprises cyclodextrin or glycerol. Such pharmaceutical compositions can be in accordance with the first aspect of the invention and can be in the form of an aqueous or a powdered composition. When the composition is aqueous, the additive can be a cyclodextrin, optionally in admixture with glycerol. When the composition is powdered, the additive is preferably a cyclodextrin. The preferred cyclodextrin is β-cyclodextrin. Such compositions are pharmaceutically acceptable because they do not cause the serious local irritation, pain and toxic side effects caused by the (poly)alcohols used in previously proposed compositions.

Nasal compositions in accordance with the invention can be administered as a nasal spray, drop, solution, suspension, gel, ointment, cream, or powder. The composition may also be administered using a nasal tampon or a nasal sponge. As previously stated, the composition is preferably administered in the form of an aqueous composition or a dry powder. The aqueous composition is preferably an aqueous solution, but can be a suspension, or a gel.

When taken as an aqueous composition suitable compositions can be obtained with or without glycerol as an additive. Glycerol allows aqueous compositions containing relatively high amounts of melatonin, does not exert toxicity towards the epithelial membrane, and does not lead to irritation of the nasal mucosa. An additional advantage of the use of glycerol is the preservative properties thereof, leading to stable solutions.

When taken as a powder, the composition preferably contains cyclodextrin and more preferably a 5-HTP-melatonin-cyclodextrin complex to obtain optimum onset times. 5-HTP-Melatonin-cyclodextrin complexes, however, can be employed in any embodiment of the present invention.

The term cyclodextrin refers to cyclodextrins such as α-cyclodextrin, β-cyclodextrin, γ-cyclodextrin, and derivatives thereof, such as methylated or alkylated cyclodextrins. Examples are methylated β-cyclodextrin, hydroxypropyl- and hydroxyethyl-cyclodextrin (di)glucosyl- or (di)maltosyl-cyclodextrins carboxymethyl- or sulfoalkyl ether cyclodextrins, such as sulfobutylether-β-cyclodextrin. The preferred cyclodextrin is methylated β-cyclodextrin, or more preferred β-cyclodextrin.

In another embodiment, preservatives may be used, referring to one or more of benzalkonium, benzalkonium chloride, potassium sorbate, benzyl alcohol, thimerosal (merthiolate), edetate disodium monobasic sodium phosphate, providone, di-basic sodium phosphate, disodium ETA, potassium phosphate monobasic, iodine, phenylcarbinol, sodium silicoaluminate, and the like. Indeed, other carriers, preservatives, buffers, moisturizers, or volatile oils or fragrances may be used in the composition of the present invention.

In another embodiment, the melatonin-5-HTP solution is preservative-free acidified solution. In an even further embodiment, the melatonin-5-HTP solution is a carrier-based solutions wherein the carrier comprises one or more of: water, pectin, oils, saline, buffers, moisturizers, fragrances, and the like.

Nasal administration may be carried out by known administration techniques and apparatus. There are various sprayers, many of which are used for delivering other medications or chemicals that would be expected to suffice for administration of the disclosed composition i.e. flexible plastic sprayer; atomising device; pump atomiser; drops; insufflator. In an exemplary embodiment the spray is administered via a metered-dose manual pump spray unit.

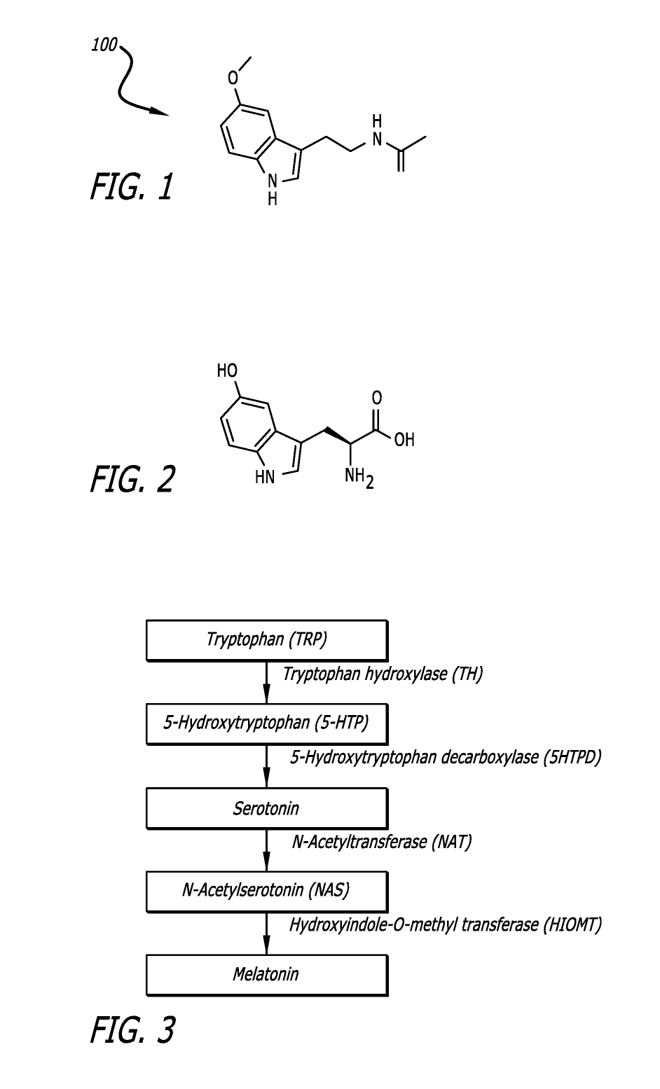

Turning to the figures of the present invention, various aspects are described. FIG. 1 provides a standard compound for melatonin 100. FIG. 2 provides a standard compound for 5-HTP. FIG. 3 describes further the metabolism pathway for a person's natural production of melatonin. A critical and essential aspect of the nature of the rapid onset (nasal administration) and short-lived effects (stimulation of natural production) lie in the synergistic, yet independent, activity of the combined melatonin-5-HTP composition, said composition being effective to cause the blood plasma concentration of melatonin in a human to increase via the rapid administration of the melatonin in a nasal spray, while simultaneously causing an increase in 5-HTP present in central nervous system tissue to increase natural production of serotonin. The combined effects of the dually administered ingredients provide a rapid increase in melatonin within the blood plasma via nasal administration while triggering increased serotonin production by administering the 5-HTP within the nasal administration. The dual effects of the two ingredients result in a rapid, yet short term increase in melatonin and serotonin production, which enhances the anti-anxiety and anti-insomnia effects of the administered compositions.

In another embodiment, the composition of the present invention may further comprise more soluble, higher weight/weight concentrations of melatonin and/or 5-HTP compositions in the form of pharmaceutically acceptable salts, while addressing risks of irritation and/or stability as aqueous solution. Additional use of volatile oils to deal with odor/taste issues or further use of a moisturizer or thickening agents may be used, including biomaterials such as hyaluronic acid and/or methyl cellulose derivatives.

In another embodiment, the composition of the present invention may further comprise a buffer system for increased stability/adjusting pH to reduce irritation. A preferred buffer system includes sodium chloride, calcium chloride, disodium hydrogen phosphate and calcium hydrogen phosphate, in a concentration sufficient to maintain the pH of the composition at a value inclusive of 5.5 to 8.5.

### Anecdotal Study of Patient Administration.

A study to assess the efficacy of a preferred composition of the present invention against multiple patients (n=11; n=12) expressing conditions ranging from insomnia to certain anxiety-based symptoms, including but not limited to: mental exhaustion, inability to sleep, anxiety, irritability, panic, frustration, or depression. The patients were instructed to administer the composition for various periods, although many of the study results were focused on acute administrations. The efficacy of the treatment of the instant composition is analyzed on the basis of patient reported outcome taken at the end of the study period. Reporting was performed via responding to a questionnaire focusing on the following criteria: administration to one or both nostrils, number of spray's per nostril, effect noticed, nature of effect, timing of first effect, nature of use—desired effect, effect of improved sleep, effect of improved relaxation, lessening of certain symptoms, and noticed effect compared to traditional oral administrations.

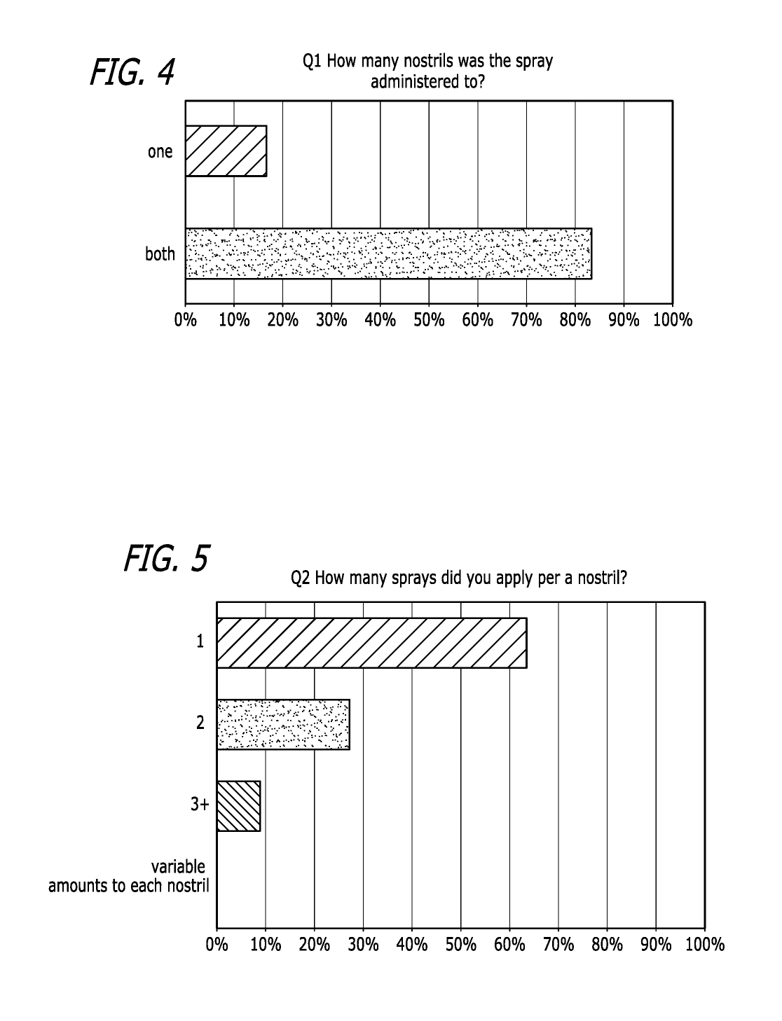

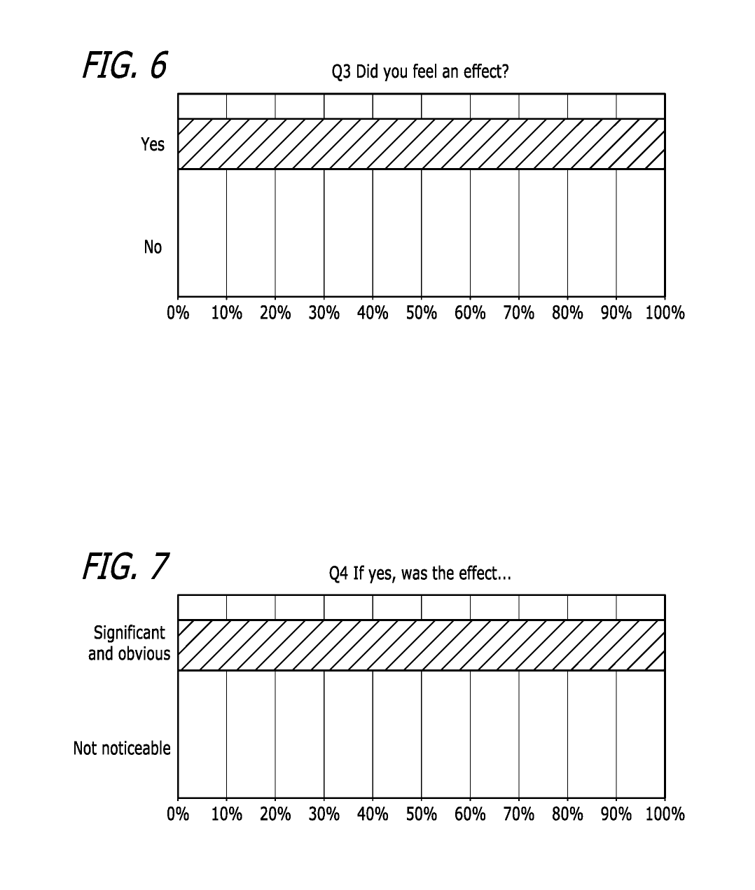

All patients administering the composition of the present invention all patients—eleven−(100%) responded as having felt an effect of the composition. Turning to FIG. 4, the only survey where 12 patients responded, 83.33% responded having administered the composition in both nostrils. The responses to the number of sprays per nostril were 64% responding once per notstril, 27% responding twice per nostril, and 9% responding 3 or more times per nostril (See FIG. 5). Turning to FIG. 6, all (100%) of respondents reported having felt an effect from administration. When queried as to whether the effect was significant and obvious, versus not noticeable, again all, or 100%, of the respondents answered that the effect was significant and obvious (see FIG. 7).

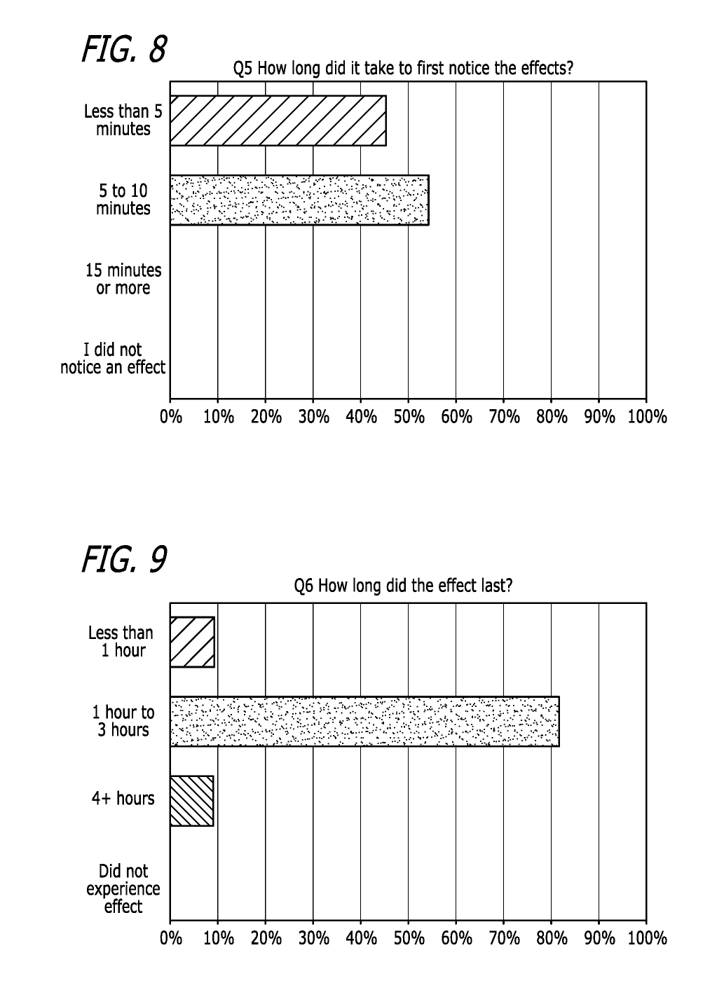

In another embodiment of the present invention, the effect of the nasal administration is ‘fast-acting’, meaning, for the purposes of the present invention, having an effect on the patient in less than 10 minutes. Turning to FIG. 8, when queried on the length of time to notice the effects of the administration, the respondent patients, 45.5% noticed effects in less than 5 minutes, and the remaining 54.6% noticed effects between 5 to 10 minutes. It is therefore an exemplary embodiment of the present invention to have a composition for intranasal administration, comprising: 5-HTP, melatonin, and a pharmaceutically acceptable carrier wherein the composition, when administered to a patient intranasally, is capable of affecting a patient in 10 minutes or less.

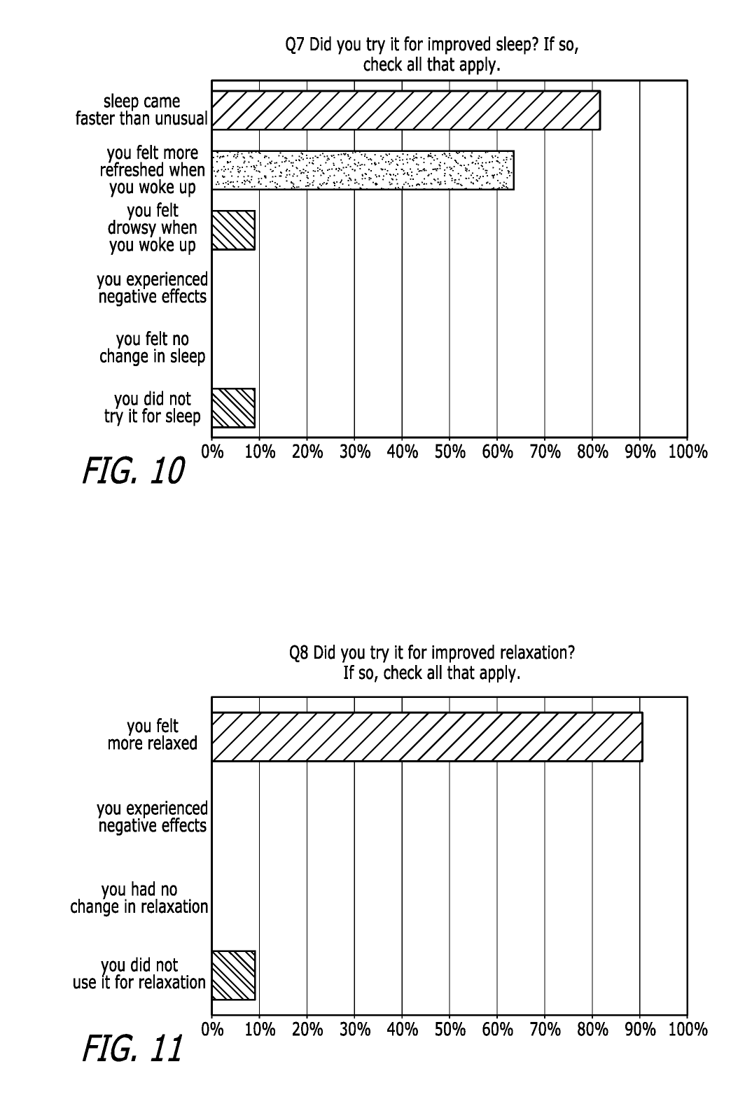

In another embodiment of the present invention, the effect of the nasally administered composition is limited, allowing for rapid metabolism. Turning to FIG. 9, when queried on the duration of the effect, 9% responded the effect lasted less than one hour; 82% responded the effect lasted between 1 and 3 hours, and 9% responded the effect lasted greater than 4 hours. In reference to FIG. 10, respondent patients (n=11) further responded to the query on whether the patient tried the composition for improved sleep. In doing so, 82% responded that sleep came ‘faster than usual’ when compared to usual circumstances without administering the composition. 63.6% of the respondent patients agreed that they felt more refreshed when they woke up after administering the composition prior to sleep. 9.1% responded that they felt drowsy when they woke up. 0% responded they felt negative effects, and 9.1% responded they did not try the composition for sleep.

Turning to FIG. 11, 90.9% of the respondent patients (n=11) responded they felt more relaxed after administration of the composition of the present invention. 0% answered they felt negative effects. 0% answered they had no change in relaxation. 9.1% answered they did not use the composition of the present invention for relaxation.

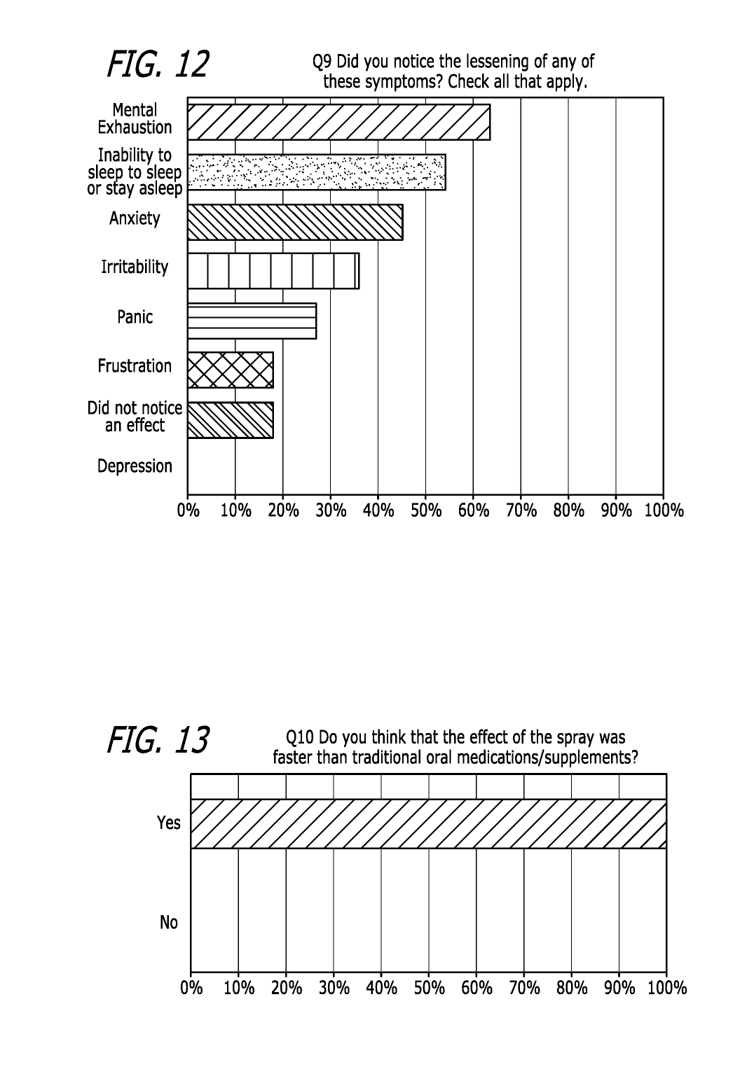

Turning to FIG. 12, the respondent patients (n=11) were queried on whether they noticed a lessening of certain symptoms. They patients were instructed to select all that applied, therefore, each percentage should be considered individually and not relative to the other conditions. 63.6% responded that mental exhaustion was lessened. 54.6% responded that inability to sleep or stay asleep was lessened. 45.5% responded they felt less anxiety. 27.3% responded they felt less panic. 18.2% responded they felt less frustration. 18.2% responded they did notice an effect on these symptoms. 0% responded regarding having noticed a lessening of depression.

FIG. 13 represents a chart showing respondent patients (n=11) responses to whether they felt the effect of the spray was faster than traditional oral medications or supplements. Of this query, 100% answered in the affirmative, supporting capability of the present invention to provide a fast-acting composition.

The above referenced figures are exemplary figures for providing illustrative aspects of the present invention, particularly within the context of the specification and its description of the claimed invention in the context of the referenced processes.

The embodiments herein provide illustrative embodiments of the present invention. While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

Those skilled in the art will recognize that the methods and systems of the present invention may be implemented in many manners and as such are not to be limited by the foregoing exemplary embodiments and examples. Furthermore, the embodiments of methods presented and described as figures in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

### REFERENCES

1. Webb S M, Puig-Domingo M. Role of melatonin in health and disease. Clin Endocrinol (Oxf). 1995; 42(3):221-234.
2. Generali J A. Keeping Up: Melatonin. Drug Newsletter. 1996; 15:3.
3. Cowley G. Melatonin. Newsweek. 1995; August 7:46.

What is claimed is:

1. A fast-acting composition for intranasal administration to a patient, comprising:
5-hydroxytryptophan (5-HTP);
melatonin; and
a pharmaceutically acceptable carrier,
wherein said composition is capable composition being effective to cause the blood plasma concentration of melatonin in a patient to rapidly increase while simultaneously causing an increase in 5-HTP present in central nervous system tissue to increase natural production of melatonin.

2. The composition of claim 1, wherein said composition is capable of affecting a patient in 10 minutes or less.

3. The composition of claim 1, wherein said composition is capable of affecting a patient in 15 minutes or less.

4. The composition of claim 1, wherein said 5-HTP is administrable in a range of 0.1 μg-650 mg.

5. The composition of claim 1, wherein said melatonin is administrable in a range of 0.1 μg-500 mg.

6. The composition of claim 1, wherein said pharmaceutically acceptable carrier is selected from the group consisting of water, saline, cyclodextrin, glycerol, or combinations thereof.

7. The composition of claim 1, wherein said pharmaceutically acceptable carrier is β-cyclodextrin.

8. The composition of claim 1, wherein said pharmaceutically acceptable carrier is selected from a group consisting of: α-cyclodextrin, β-cyclodextrin, γ-cyclodextrin, methylated β-cyclodextrin, hydroxypropyl- and hydroxyethyl-cyclodextrin (di)glucosyl- or (di)maltosyl-cyclodextrins, carboxymethyl-cyclodextrins, or sulfobutylether-β-cyclodextrin.

9. The composition of claim 1, wherein said intranasal administration is a spray, drop, solution, suspension, gel, ointment, cream, powder, nasal tampon or a nasal sponge.

10. The composition of claim 1, wherein said intranasal administration is for rapid treatment of insomnia.

11. The composition of claim 1, wherein said intranasal administration is for rapid treatment of anxiety.

12. A method of rapid improvement of sleep behavior of a patient, comprising administering to a patient via nasal administration a therapeutically effective amount of a composition comprising 5-HTP and melatonin, wherein said composition causes the blood plasma concentration of melatonin in a patient to rapidly increase while simultaneously causing an increase in 5-HTP present in central nervous system tissue to increase natural production of melatonin

13. The method of claim 12, wherein said composition further comprises a pharmaceutically acceptable carrier.

14. The method of claim 13, wherein said pharmaceutically acceptable carrier is selected from a group consisting of water, saline, cyclodextrin, glycerol, or combinations thereof.

15. The method of claim 12, wherein said rapid improvement is initiated in 10 minutes or less.

16. The method of claim 12, wherein said composition is administered in the form selected from a group consisting of a spray, drop, solution, suspension, gel, ointment, cream, or powder, nasal tampon or a nasal sponge.

17. A method of treating anxiety of a subject, comprising administering to a patient via nasal administration a therapeutically effective amount of a composition comprising 5-HTP and melatonin, wherein said composition causes the blood plasma concentration of melatonin in a patient to rapidly increase while simultaneously causing an increase in 5-HTP present in central nervous system tissue to increase natural production of melatonin.

18. The method of claim 17, further comprising administering said composition having a pharmaceutically acceptable carrier selected from a group consisting of water, saline, cyclodextrin, glycerol, or combinations thereof.

19. The method of claim 17, wherein said composition is administered in the form selected from a group consisting of a spray, drop, solution, suspension, gel, ointment, cream, or powder, nasal tampon or a nasal sponge.
